(19) United States
(12) Patent Application Publication
(10) Pub. No.: US 2004/0111079 A1
(43) Pub. Date: Jun. 10, 2004

(54) Targeted sanguinous drug solution delivery to a targeted organ

(76) Inventors: Richard Hayes, McKinney, TX (US); Jakob Vinten-Johansen, Grayson, GA (US)

Correspondence Address: CARSTENS YEE & CAHOON, LLP, P O BOX 802334, DALLAS, TX, 75380

(21) Appl. No.: 10/726,463

(22) Filed: Dec. 3, 2003

Related U.S. Application Data
(60) Provisional application No. 60/430,544, filed on Dec. 3, 2002.

(51) Int. Cl. A61K 9/22

(52) U.S. Cl. 604/890.1

(57) ABSTRACT

A system and method for precisely controlling the delivery of two fluids to a specific organ or region of a patient when one of the fluids breaks down the other fluid. The fluids are separately metered and are maintained in separate delivery lines until a point very near the target organ, preferably within twelve inches of the delivery site. In an exemplary embodiment, blood and a cardioplegic agent are supplied to the heart via the coronary arteries or coronary veins while adenosine, which provides a powerful protective influence on the myocardial cells during ischemia, is provided in a separate delivery line to prevent the breakdown of adenosine, which has a half-life of 13 seconds in the blood.

### CROSS-REFERENCE TO RELATED APPLICATION

This application claims the benefit of and priority to a U.S. Provisional Patent Application No. 60/430,544 filed Dec. 3, 2002, the technical disclosure of which is hereby incorporated herein by reference.

### BACKGROUND OF THE INVENTION

1. Technical Field

The present invention relates to the delivery of precisely measured drugs in a precisely measured volume of blood to a targeted organ. More specifically, it relates to such a delivery when the drug has an extremely short half-life in the blood, reducing the drug's concentration, efficacy, and therapeutic benefit.

2. Description of Related Art

Currently, medications can be delivered to the body by many routes, including oral ingestion, inhalants, transdermal (through the skin) transfer, intra-cutaneous (into the skin) injection, intra-vascular (into the blood vessels) administration, direct delivery of the drug via a catheter, direct injection to an organ, delivery via an extracorporeal (outside the body) circuit, and an implantable drug pump. For some surgical procedures, it is highly desirable to deliver the medication directly to a targeted organ or region. There can be one or more drugs, which must be precisely given, either together or in a particular sequence, to the target anatomical structure over a period of time.

The Multi-Pump System (MPS) of Quest Medical Inc. was originally developed to serve such a need, specifically the delivery of a measured amount of blood containing a precisely measured amount of a cardioplegic medication (to temporarily paralyze the heart muscle) to the vascular system of the heart during open-heart surgery. It was, and continues to be, used in conjunction with a heart/lung machine, which handles total blood flow to and from the body while the heart is stopped, as well as finding a place in newer procedures where delivering medicated blood solutions may be beneficial to the patient.

FIG. 1 shows a diagrammatic representation of the connections to the heart during open-heart surgery using a heart-lung machine and an MPS to handle blood flow. In this diagram, the heart 100 is seen, as well as a number of the major arteries and veins connected to the heart. These include the superior vena cava 110 and inferior vena cava 112, which return oxygen-depleted, carbon dioxide-rich blood to the right atrium of the heart 100. From there, the blood normally goes to the right ventricle, so that it can be sent to the lungs through the pulmonary artery 114. Once the oxygen and carbon dioxide have been exchanged in the lungs, blood normally returns to the heart through the pulmonary vein (not visible in this view) to the left atrium, then to the left ventricle, where it is sent out to the entire body through the aorta 116. The coronary arteries 118, which supply blood to the heart itself, come off the aorta 116 at its root. During open-heart surgery, a tube 120 can be connected to the right atrium of the heart, where it receives the venous blood returning to the heart. This blood is sent to the heart/lung machine 130, where it is oxygenated and carbon dioxide removed, causing the blood to become arterialized again. The temperature of the blood can also be adjusted at this time. The majority of this blood is returned directly to the body through tubing 122, connected here to the aorta 116. The remainder of the blood, about 10 percent, is passed to the MPS system 140, where precise amounts of cardioplegia medications are added to the blood before it is returned to the coronary arteries 118 through tube 124. An alternative route of delivery of is through the coronary sinus in a retrograde (backward) flow. It is necessary for the circulation handled by this machinery to be constantly monitored during surgery. In order not to interfere with the actions of the surgeon, the heart-lung and MPS machines are located outside the surgical field. The various tubes 120, 122, 124 are bundled together and led to the appropriate mechanical connections. This tubing and the parts of the circuit passing through the various machines is primed with priming solutions which may contain blood prior to the operation.

The drawing illustrates only one possible connection between the system and the patient. In an alternate embodiment, the blood can be removed by a catheter that is passed up through a vein until it reaches the junction of the vena cava with the right atrium, with blood restored to the aorta through a separate catheter inserted, for example, into the femoral artery and fed into the aorta.

Turning now to the more mechanical side of the drawing, tubing 120 from the right atrium is in fluid communication with the arterial pump 132 of the heart-lung machine 130, which draws blood out of the body. The arterial pump 132 forces the blood through an oxygenator/heat exchanger 134, which acts as an artificial lung to exchange oxygen and carbon dioxide, then through an arterial filter 136. A heater/circulator 138 provides water at a predetermined temperature for the heat exchange portion of the oxygenator/heat exchange 134 so that the blood can be brought to a desired temperature by heat exchange with the water. A venous reservoir 131 is normally connected between the arterial pump 132 and the patient to store and maintain sufficient fluid volume for proper operation. After passing through arterial filter 136, most of the blood returns to the body via tubing 122. The MPS system 140 receives a portion of the oxygenated blood for processing and returns it to the body through tubing 124. Valves 133, 135 can be opened to fill the apparatus with blood or to adjust the volume in the circuit, while sensors 137 measure pressure at various points in the circuit and provide feedback to controller 139, which is also connected to the arterial pump 132. Connections between the sensors 137 and the controller 139 are shown as dotted lines.

Further details of the MPS system 140 are seen in FIG. 2, which is adapted from U.S. Reissue Pat. No. 36,386, which is owned by the assignee of this application and which is hereby incorporated by reference. This figure depicts a prior art cardioplegia delivery system 140 (shown in FIG. 1), established to provide a mixture of blood and a cardioplegic solution to the heart of a patient during open-heart surgery. The mixture is delivered to the system through a conduit 212 that is connected to the output of the heart/lung machine 130, which provides oxygenated blood through the main extracorporeal return line 122 to the patient. The fraction of the blood supply designated for the heart is diverted into conduit 212 for processing by the cardioplegic circuit and delivery to the patient's heart through line 124. The cardioplegic solution flowing through line 124 is delivered through an antegrade line (i.e., in the normal direction) to the coronary arteries or through retrograde line (i.e., in the reverse direction) to the coronary sinus, as required by the surgeon.

A crystalloid solution is stored in container 224 for combination with blood flowing in line 212 at a disposable pumping cassette 226. The output of cassette 226 is supplied through line 228 to a heat exchanger 231. Pump cassette 226 is controlled by an electromechanical pump mechanism 230 in which cassette 226 is mounted. A second pump 232, containing a cardioplegic agent such as a potassium solution, supplies its output to line 228 downstream from the pump cassette 226. A third pump 241 may also be included to supply any variety of additives as may be desirable for a particular operation or as may be otherwise requested by the surgeon or by the operating team. The output will be injected into line 228 downstream from cassette 226.

Preferably, pumps 232 and 241 may be syringe pumps or volumetric pouches of a type well known in the infusion art. In the case where pump 232 is a syringe pump, a solution containing a heart arresting agent such as potassium may be loaded into a syringe, and the syringe mounted in pump 232 which progressively depresses the syringe plunger to deliver potassium solution to line 228. The flow rates of potassium solution are less than about 10%, and preferably less than about 5%, of the total flow rate issuing from pump cassette 226. An accurately controllable pump, such as a syringe pump, may be advantageously used in applications where a particular fluid additive or constituent must be an accurately controlled small portion, less than about 10%, of the total flow volume. Similarly, other additives will typically be limited to a small percentage so that accurate control on pump 241 is advantageous.

In the heat exchanger 231, the cardioplegic solution is juxtaposed with a circulating, temperature-controlled fluid to adjust the temperature of the solution prior to forwarding the solution to the heart through line 218. Preferably pump 233 circulates temperature-controlled fluid through the heat exchanger 231 either by push or pull. In this example, a push through coolant system utilizes a pump 233 to circulate a control fluid through heat exchanger 231 and then to a two-way valve 234. Valve 234 directs the control fluid either to an ice bath 235 for cooling or a heated water reservoir 238 for heating. The control fluid is then pumped via valve 240 back through the heat exchanger 231 where the cardioplegia solution receives heating or cooling without contamination across a sealed heat transfer material or membrane within the heat exchanger 231.

The system includes patient monitoring of myocardial temperature along the signal path 242 and heart aortic root pressure along signal path 245 or coronary sinus pressure along signal path 244 communicating to a central microprocessor control section 246. In addition, the pressure and temperature of the cardioplegic solution in delivery line 218 is sensed and the data is forwarded along signal paths 248 and 250 to the control microprocessor 246. Data input to microprocessor 246 through control panel 252 may include an advantageous combination of the following parameters:

1. Desired overall volumetric flow rate through disposable pump cassette 226.

2. Desired and measured pressure of the cardioplegia fluid delivered to the patient.

3. Desired blood/crystalloid ratio to be forwarded by disposable pump cassette 226.

4. Desired potassium concentration to be established by pump 232.

5. Desired and measured temperature of solution in cardioplegia delivery line 218.

6. Safety parameters such as the pressure of the cardioplegia solution in the system or upper and lower limits for pressure in the patient.

In response to the data input through the control panel 252 and the monitored conditions along the signal paths 242, 243, 244, 245, 248, 250, microprocessor control section 246 controls the operation of the pump mechanism 230 via a first signal path 254, and of potassium syringe pump 232 via a second signal path 256. The control signals for a third pump 241 for additives may be communicated along path 257 between the control section 246 and pump 241. In addition, microprocessor control section 246 controls the circulation of fluid in the heat exchanger circulation path along signal path 258 either for obtaining a desired patient temperature or a desired output solution temperature. Further, the safety parameters such as pressure limits for a particular procedure or a particular patient may be controlled based upon input settings or based upon preset standards, as for example, one range of acceptable pressure limits for antegrade and another range for retrograde cardioplegia. The ranges may be set by the operator or may be set automatically based upon preprogrammed default values or may be calculated based upon preprogrammed algorithms in relation to a selected desired patient delivery pressure.

Communication connections or signal pathways 242, 243, 244, 245, 248, 250, 254, 257, 258 and any others as may be appropriate can be electrical signals through conducting wires, light signals through optical fibers or transmitter radio, ultrasonic or light signals.

In accordance with the invention, the microprocessor controller section 246 controls the pump mechanism 230 to combine crystalloid from container 224 and blood from line 212 in any selected ratio over a broad range of blood/crystalloid ratios. The controller 246 may command the pump mechanism 230 to deliver blood without crystalloid addition. A preferred range for the blood/crystalloid ratio adjustment capability is from 0 to 20:1 or all blood. The rate of flow produced by the pump mechanism 230 of the combined output from disposable pump cassette 226 is preferably variable from 0 to 500 milliliters per minute. The pump mechanism 230 may be operated by microprocessor 246 in either a continuous or intermittent mode by instruction through control panel 252. The arrest agent syringe pump 232 is automatically controlled to deliver at a rate such that the introduction of an arrest agent, such as a potassium solution, to line 228 is automatically maintained at the selected concentration vis-a-vis the flow of disposable cassette 226, without regard to changes requested in the flow rate from pump cassette 226 or changes in the blood/crystalloid ratio, requested of the pump mechanism 230 through microprocessor 246. The operator may directly request flow rates using the control panel.

Some of the desirable features of the MPS system will now be summarized. Those desiring further information regarding how these features are implemented are referred to Reissue Pat. No. 36,386, referred to earlier.

First, the system is modular and configurable. This allows a perfusionist (a medical technician responsible for the extracorporeal oxygenation of blood through the operation of the heart-lung machine and MPS system) to monitor the administration of a number of medications for the heart through a single system. As new medications are introduced for surgeries, the machine can be adapted to handle additional pumping assignments.

Second, many facets of operation are handled automatically, while giving the operator the ability to change its operation. For example, in many heart surgeries, the blood is mixed with a crystalloid solution within the main pump. The ratio of blood to crystalloid solution is variable over a wide range, settable by the operator. However, once the ratio has been set, it is maintained automatically, without further operator intervention, unless a change is requested. Similarly, a separate pump is used to deliver the cardioplegic solution, but its delivery rate can be set to maintain a fixed, but changeable ratio to the delivery rate of the blood solution. In a similar manner, other solutions to be added can be separately metered in a settable relationship to the blood flow.

The MPS system uses internal monitors, as well as monitors on the patient, to provide feedback to conditions such as temperature, pressure, concentration of a factor in the blood, etc. The system can respond to conditions received by the monitors, for example, by altering the pumping speed to maintain the blood pressure at a desired level. As well as controlling operations within the system, the processor will alert the perfusionist to changing conditions that may indicate developing problems. Current conditions received from the monitors are displayed on the display face.

The operator can also change desired conditions as the operation progresses. For instance, it can be desirable to cool the blood to a constant temperature during the operation, warming the blood back to normal body temperature as the operation concludes. Using the MPS system, the perfusionist can indicate the desired temperature. This prompts the processor to determine whether the blood needs to be brought in contact with a heated water bath or a cooling water bath and to change the valve appropriately, as well as to set the thermostat of the water bath to the desired temperature.

Third, the system is configured to be as intuitive as possible. A perfusionist needs to be able to take in any pertinent facts about the patient and the system very quickly in order to be able to respond with the necessary speed. Both the display and the controls are arranged logically and systematically to make this easier. Essential information is displayed more prominently, so that the user's attention is easily drawn to the most vital information.

Disposable pump cassette 226 is illustrated in FIG. 3. The cassette may be formed from two flexible plastic sheets 360 bonded together selectively to form open flow paths and chambers therebetween. Each sheet 360 may be of any simple flexible material such as polyvinylchloride, and the sheets may be radio frequency welded together, leaving the flow paths and pump chambers unbonded. A bladder cassette of this type advantageously reduces the shearing forces and potential damage to which blood might be subjected in other pumps, such as peristaltic pumps.

The entry side 362 of the cassette 226 includes a blood inlet 364 and a crystalloid inlet 366. Inlets 364 and 366 lead to a common pump inlet path 368, which is bifurcated to form two pump flow paths 370 and 372. The first flow path 370 leads to an enlarged fluid bladder pump chamber 374, while the second flow path 372 leads to an identical fluid bladder pump chamber 376. The two outlet paths 378, 380 from their respective pump chambers 374, 376 are joined at a common outlet 382 from cassette 326 for delivery of the mixed cardioplegic solution to the output line 228.

FIG. 3 depicts six valve sites 384, 386, 388, 390, 392, 394 located along the fluid paths in cassette 326 according to one embodiment for which the invention is useful. These are sites at which the corresponding flow path may be occluded through operation of a valve plunger on the pump mechanism 230, to press the sheets 360 together at the valve, when the cassette 326 is mounted in operating position in the mechanism 230. In this embodiment, a first valve 384 is positioned to occlude the outlet path 378 from the first pump chamber 374. A second valve 386 is positioned to occlude the outlet path 380 from the second pump chamber 376. Bladder inlet valves 388, 390 are placed along the pump chamber inlet paths 370, 372. The final two exemplary valves 392, 394 control the passage of blood or crystalloid alternately to their common inlet path 368 are positioned at their respective inlets 364, 366.

One embodiment of a pump mechanism 230 is illustrated in FIG. 4, and incorporates a pair of pumping motors 495 and 496. A first pumping motor 495 is positioned to advance and retract a bladder-driving element 498, and a second pumping motor 496 is positioned to similarly operate a second bladder-driving element 400. A valve cam motor 402 is provided to operate all valve closures on the disposable cassette 226. The cam motor 402 turns an inlet camshaft 404 carrying valve-cams 406, 408, 410 and 412. The camshaft 404 also turns, by means of pulleys 414, 415 and a timing belt 416, an outlet camshaft 418. Outlet camshaft 418 carries two valve-cams 420, 422.

As best seen in FIG. 5, disposable cassette 226 is positioned tightly against the face of pump mechanism 230 by a closing door 524 so that the cassette bladder pumping chambers 374 and 376 are enclosed, and confront driving elements 498 and 400. Driving elements 498 and 400 may be of identical construction, and preferably of the petal module type disclosed in U.S. Pat. No. 4,657,490, the disclosure of which is incorporated herein by reference. Although driving elements of this petal module type have the advantage of a linear relationship between displacement by the pump motor and volumetric displacement from the pump chamber, by their close compliance and confrontation to the plastic disposable cassette and by reduced shearing forces associated with the smooth pump action, other driving elements which provide a predictable volumetric displacement by a given advancement of the motor might be utilized.

The variable surface area type of driving element illustrated includes a hub 530, surrounded by radially extending, pivotally mounted petals 532 so that the hub 530 together with the petals 532 provides a confronting surface for the confined pump chamber. Advancement of a motor 495, 496 causes its hub 530 to advance and carry the petals 532 along with it to reduce the volume of the confined pump chamber. Conversely, retraction of a motor 495, 496 causes the corresponding driving element 498, 400 to retract, withdrawing the constraint on chamber volume.

In FIG. 5, element 498 is illustrated substantially fully retracted, so that pump chamber 374 is filled with fluid, and element 400 is pushed to its full advancement, emptying its pumping chamber 376. Means for measuring the force necessary to advance each of the motors, or a pressure sensor contacting the cassette 226 (not shown) is also provided to enable microprocessor 246 to record data representative of the pressure on each bladder chamber.

FIG. 6 illustrates the valve action embodied in mechanism 230, by showing the inlet and the outlet valve arrangement from a single pump chamber. All six valves 384, 386, 388, 390, 392, 394 and their respective valve cams 406, 408, 410, 412, 420, 422 operate in similar fashion. Driving element 530 engages the disposable pump chamber 374. Inlet plunger valve 388A and outlet plunger valve 384A, controlled by cams 412 and 420 are normally closed by the action of biasing springs 634 and 636. In the closed condition dictated by the biasing springs, each valve plunger presses against its corresponding valve site on disposable cassette 226 closing the corresponding fluid path. Each valve site 384, 386, 388, 390, 392, 394 is provided with a similar, normally closed valve. Each of the valve sites 384, 386, 388, 390, 392, 394 is opened under the action of valve cam motor 402 upon rotation of its corresponding cam to an open position, retracting the valve plunger from the disposable cassette, and opening the corresponding flow path flow. In FIG. 6, the cam 412 has moved to the open position, retracting the valve plunger 388A to open the valve 388 on the cassette 226, opening the inlet 370 of bladder chamber 374 for entrance of fluid.

It will be appreciated that a change in the ratio of blood to another constituent, such as crystalloid, is a simple adaptation for the pump mechanism 230. A change to the ratio is requested through control panel 252 and microprocessor 246 directs the motors 495 and 496 to retract by different amounts during their blood-fill and crystalloid-fill steps. The full retraction of a motor is the same for the combined fill. It is simply necessary to adjust the amount of retraction during each fill step to the requested ratio. The ratio may be continuously adjusted from 100% of blood to 100% crystalloid. Thus, if the requested blood/crystalloid ratio is R, and the motor driven-volume displacement relationship is linear, then R=(Number of motor steps retracted during blood fill)/(Number of motor steps retracted during crystalloid fill).

The total volumetric flow rate from the cassette is varied pursuant to operator request simply by compressing or expanding the time for a cycle to be completed. Of course, if intermittent operation is desired, this may be provided as well.

No matter what changes may be made to the blood/crystalloid flow rate, microprocessor 246 preferably automatically controls the arrest agent pump 232 to deliver at a rate which provides the requested percentage of the then-existing blood/crystalloid flow rate.

One or more other additives may be added to the blood mixture fluid as with an additive pump 241, which is controlled from control panel 252 through microprocessor 246 and along signal path 257. Typically, any combination of additives may be premixed for insertion through one additional pumping mechanism 241, although another could also be incorporated in a similar manner, separately controlling the amount of individual constituents or additives. As with pump 232, the ratio can be automatically maintained according to the flow rate of pump 230. This advantageously facilitates the capability of this mechanism to function in an automatic constant pressure mode, where the flow rate may be continuously varied to maintain a constant pressure according to the present invention.

FIG. 7 is a detailed perspective view of a preferred embodiment of control panel 252. Control panel 252 has a front face 740 which is viewable from a wide frontal angular area, including substantially 180.degree. In the preferred embodiment, a substantially flat face 740 works well and is constructed using standard molding techniques, stamping techniques and components. Advantageously, a flow path 742 is visually depicted on the front panel interconnecting with portions of the substantially visually continuous flow path interconnecting two or more system component display areas, as with interconnecting portions 742a, 742b, 742c and 742d. Preferably, the two or more system components are those which are those system components which represent characteristic elements of the system which are adjustable through controls interconnected with the control panel 252, such as through a microprocessor control section 246, shown in FIG. 1. Also preferably, the visual depiction of the flow path 742 is formed with sufficient width and having sufficient contrasting color between the flow path 742 and the face 740, as for example, with a red flow path line 742 on a white or light beige or light gray background base 740. A width of approximately 3/16 of an inch to 5/16 of an inch (about 0.5 cm to 0.8 cm) with a bright oxygenated blood red color on a light gray background has been found to be easily visually perceptible from normal viewing distances in an operating room, it being observed that the normal maximum distance which the perfusionist is likely to move from a control panel during an operation will be about 9 to 15 feet (about 3-5 meters).

In the preferred embodiment, the flow path is provided with a start indicator, such as an arrow or arrowhead 744, which may be illuminated when the system is in an “on” position. Also, the flow path 742 is provided with a depiction of the delivery and of the flow path, as with a depiction of an organ, limb or other part of a patent, such as a heart 746, at the opposite end of the flow path from the start 744.

One of the first components which has desirably adjustable characteristics for the perfusionist is a blood-to-crystalloid ratio display area 748, which includes an adjustment actuation button 750, a digital display 752, a dynamic pump action display 754, a label 756 associated with the digital display 752, and the dynamic pump action display 754 so that the operator will immediately understand which component of the system is represented by those displays within area 748. Whenever the pump is operating, display 754 is animated to show up and down pump action so that the operator immediately recognizes whether the system is operating. Upon depressing adjustment button 756, the set mode is actuated for establishing a desired blood-to-crystalloid ratio. Preferably, button 750 becomes lighted to indicate it is in an adjustment mode or a “set-up” mode and the digits within digital display 752 become brighter so that the operator is immediately notified that the blood-to-crystalloid ratio is in a condition for being set. Also, a set indicator light 758 display is comes on or is otherwise lighted and the adjustment knob 760 is activated for manually adjusting the desired blood-to-crystalloid ratio, which adjustments will be continuously displayed within digital display 752. Once the desired ratio is established, then the operator again toggles the button 750 so that it is in an out position, turning off the light therebehind, dimming the digital display 752 and disconnecting knob 760 so that the set light 758 goes off.

The operation of the adjustment knob 760 in connection with setting various ones of the adjustable parameters of the system will be explained more fully below. For a preliminary understanding, there are various adjustment actuation switches or buttons that are associated with the display areas. These switches can periodically engage the set knob 760 to adjust components of the system. These components do not necessarily require adjustment for each patient so that a single adjustment knob 760 can be used with separate components while the others are maintained at a previous setting.

Flow rate display area 762 includes a digital display area 764 and a continuously engaged flow rate adjustment knob 766. The flow rate display area 762 also includes a label 768 adjacent to the digital display 764 so that the operator, perfusionist or surgeon immediately associates the digital display with the appropriate adjustable characteristic or parameter of the system. As the flow rate is typically the primary variable feature with respect to each patient, the adjustment knob 766 is continuously engaged and does not require actuation of an adjustment switch in order to engage the adjustment knob. The perfusionist may variably dial in the flow rate as required for each patient. It will be seen in the embodiment depicted in FIG. 7, flow rate area 762 follows closely adjacent to the blood-to-crystalloid ratio display area 748 along flow path 742. The flow rate controls the rate of pumping. It is positioned on the display through a visual and logical correlation to the system which is understandable by the perfusionist and which reduces confusion and facilitates quick reaction by the perfusionist to any changing conditions during surgery. Normally, the perfusionist gradually increases the flow rate from a low initial value up to a desired pressure value, while watching an indicator of the pressure of the cardioplegia fluid at a catheter interconnected with the heart. The desired pressure will depend upon overall considerations, including whether the system is being operated in a retrograde flow or an antegrade flow direction. The perfusionist typically approaches the desired pressure slowly so that damage to the blood vessels supplying the heart with cardioplegia fluid is avoided. A constant pressure can be defined by selecting an automatic constant pressure mode of operation when the desired pressure is reached by manually adjusting the flow rate.

In normal cardioplegia delivery, an arrest agent will be added to the cardioplegia fluid at one high level of concentration initially in order to stop the heart from beating and subsequently after the heart has been sufficiently stopped from beating, will be maintained in an arrested condition with a low concentration of the arresting agent in the cardioplegia solution. Correspondingly, on the control panel 752 of FIG. 7, the arrest agent display area 770 preferably includes an arrest agent adjustment switch 772 which may be a depressible two position switch and also a high or low concentration selection switch 774, both of which can be activated to engage adjustment or set knob 760 and cause the set light 758 to light up. The digits in digital display 778 will also become brightened when the adjustment switch 772 is activated. When the value of the arrest agent concentration displayed in digital display 778 is greater than zero, then an on indicator light 782 will become activated. Preferably, the on light is in the shape of an arrow or arrowhead, which visually conveys the concept that an arrest agent will be entering the tubing 124 which will be carried to the heart 100 of the patient. Uniquely, the high concentration or high amount of arrest agent (i.e., the amount or mixture which will stop an initially beating heart) can be adjusted separately from the adjustment of a low concentration merely by pressing or toggling the high or low selection switch 774. The different concentrations can also be selected for delivery to the patient by merely pressing or toggling the high or low selection switch 774. After the heart is stopped with a high concentration, a lower concentration of arrest agent will maintain the still heart. The perfusionist can adjust the low level of arrest agent separately and then during operation can select a low arrest agent supply to the patient. Switching from high to low and back again is advantageously a one-button procedure.

At any time before or after the blood-to-crystalloid ratio is established and a flow rate begins with or without an arrest agent, a surgeon may determine that an additional additive should also be included within the cardioplegia solution. For this purpose, the additive may include one or more medicinal solutions or compositions and the option for controlling the addition of this additional additive is provided with a display area 784, including an adjustment activation switch 786, a digital display 788 and an on or additive included light 790. When the value in display 788 is zero, the light 790 is off and when it is greater than zero, then light 790 comes on to indicate to the perfusionist and those observing the control panel display that an additive is being included.

Once the solution is complete as to its composition, then it will be heated or cooled depending on the requirements of the particular phase of the heart operation. Typically, during a myocardial procedure, the heart will be cooled with a cold bath during the operation and will be warmed subsequent to the operation in order to revive operation of the heart. Depending on the protocol of the operation involved, various phases of heating and cooling of the heart may be required. The heat exchange or display area 792 includes a switch 794 by which the temperature of the warm bath or the temperature of the cold bath may be alternatively detected and viewed at display 796, which is associated with an understandable label 798. A delivery temperature adjustment switch 700 is provided which upon depressing engages the set knob 760 and lights up the set light 758 to adjust the desired delivery temperature that is display in a digital display 702. A label 704 is provided adjacent the digital display 702 and preferably, is on or associated with the adjustment switch 700 which indicates that this digital display is representative of the delivery temperature. Again, when the delivery temperature adjustment switch 700 is activated, it will become lit and digital display 702 will increase the light intensity so that the perfusionist will immediately understand that the adjustment knob 760 is directed to the delivery temperature.

The system pressure is supplied at a system pressure display area 706, which is provided with a digital display 708 and a label 710. Normally, the system pressure depends upon the flow rate and the patient delivery pressure, and also upon the particular configuration of the system. An inordinately high system pressure can indicate a kink, bend, or blockage in a tube or other potential problems. For example, where the system pressure is substantially higher than the patient delivery pressure, then in that event, there may be a risk that through movement of the delivery tubing or the delivery catheter, an obstruction may be alleviated which will result in an excessive system pressure temporarily becoming a potentially dangerous excessive patient delivery pressure. The perfusionist can be on guard for such a situation and can thus be ready to respond for the safety of the patient.

A preferably adjustable key characteristic or parameter of the system is the patient delivery pressure. This may be measured at a catheter or cannula at which the system is connected to the patient's blood vessels. A read-out of the patient delivery pressure is included within a delivery pressure display area 712. A digital display 714 with an appropriate label 716 is provided. Preferably, both the flow rate display 764 and the delivery pressure display 714 are positioned centrally located for ease of observation and the attention of the perfusionist, as they are substantially key characteristics of the system. Also preferably, the flow rate display 764 and the pressure display 714 are larger than the other characteristic displays so that attention is immediately drawn to these features without undue “hunting” by the operator.

Surgeons change the direction of delivery (antegrade or retrograde) to achieve optimum distribution of cardioplegia solution. The pressure must also be adjusted accordingly. Because of the different delivery scenarios, it is advantageous to have a system control panel that is intuitive by logical depiction of the system flow paths. Establishing a defined pressure and flow rate for the particular setup, whether antegrade or retrograde, is facilitated by clear visual depiction of the flow direction. Once the appropriate flow and pressure are established, as through slowly increasing the flow until the appropriate pressure is reached, then the system can be switched to a constant pressure mode to continue optimum delivery to the patient.

A visual display is provided in which indicators 720 and 722, such as an indicator light 720 indicating retrograde flow and an indicator light 722 indicating antegrade flow will be activated by the perfusionist depending upon the system connections and catheterization of the patient. There is also a retrograde adjustment switch 724 and a retrograde flow “on” light 726, as well as an antegrade adjustment switch 728 and antegrade flow “on” indicator 730. In a preferred embodiment, flow lights 726 and 730 are dynamic or animated indicators which have flashing or a sequentially illuminated series of lights which give the appearance of movement toward the heart along the flow path corresponding to the operating mode of the system at the time. If the flow stops, the dynamic lighting or animation of flow also stops; this condition is immediately perceivable by the perfusionist or the surgeon.

In a basic mode the antegrade and retrograde switches 224, 228 are provided so that the perfusionist can select from the panel the flow direction to be displayed. The selection of the flow direction may depend upon the indication from the surgeon which direction is activated by the surgeon. Activation of the switch by the perfusionist will activate different sets of default limits and alarms and as the delivery pressure displayed at 714 is typically a reading which is detected at the entry catheter, whether in the aortic root or in the coronary sinus, so that appropriate input to the display 714 is determined by selection switches 724 or 728.

During surgery, it is advantageous to continuously monitor the operation of the system. It is also advantageous to allow the perfusionist a certain degree of freedom to attend to various matters, such that alarm limits may be set. A limit display section 732 is advantageously provided in which an upper limit display 734 and a lower limit display 738 are provided. Initially, the upper and lower limits are set by default or by the perfusionist to establish maximum and minimum safe patient delivery pressure. The actual pressure corresponding to the patient delivery pressure at display 714 and the actual flow rate to the patient is advantageously depicted with an analog pressure display 731, which is positioned between the upper and lower limit digital displays 734 and 738. The perfusionist can visually observe the relationship of the patient delivery pressure as digitally displayed at display 714 in relationship to the upper and lower limits 734 and 738. A lighted label 733 is provided in the analog display area 731 to clearly indicate which limits are being observed.

The safe limits will be different for antegrade flow or for retrograde flow directions. Setting limits separately, depending upon flow direction, may be accomplished with retrograde adjustment switch 735 and with antegrade adjustment switch 737. Depression of either switch 735 or 737 will activate the set knob 760 so that the upper and lower limits can be adjusted for each flow direction. It is noted that the operator may view the limits separately for the antegrade and the retrograde flow direction. As shown more clearly with reference to FIG. 8, the same display area upper limit 734 and lower limit 738 can be used in connection with a flow rate limit display in which an analog display 749 of the actual flow rate is provided and has a lighted label 751 to clearly indicate that the upper flow rate limit 753 is activated and the lower flow rate limit 755 is activated. Again, the upper and lower flow rate limits can be separately set by the operator or by the control section 246 for retrograde and for antegrade flow through the patient's heart. The antegrade switch 737 and retrograde switch 735 may be used to separately display and/or set the limits. In the normal operation of cardioplegia delivery, the perfusionist has control over and adjusts the flow rate with knob 766. This condition is preferably the default condition during the normal run mode.

It has been found advantageous, during surgery and during continuous operation of a cardioplegia delivery system in the run mode, to maintain a defined constant pressure. As used here, delivery at a constant pressure means more than simply avoiding an upper limit. Adequate flow also requires keeping the pressure above a certain lower limit. Delivery at a constant pressure addresses both avoiding potentially unsafe high pressure and also inadequate low pressure. Delivery to the target tissue is optimized at a defined constant pressure within the range of a safe upper limit and adequate delivery lower limit. As pressure for a given cardioplegia system is dependent upon and proportional to the flow rate, automatic microprocessor control of the flow rate can be programmed in order to maintain a defined pressure. Some of the advantages of a constant pressure delivery system include the prevention of excessive pressures that can cause physical damage to the heart while keeping the capillaries expanded or dilated for optimum delivery. The use of upper and lower flow rate limits ensures adequate delivery to the heart tissues when a constant pressure is maintained. For example, it is not unusual for the retrograde cannula to become dislodged from the coronary sinus, resulting in delivery of the cardioplegia solution to the right atrium rather than to the heart tissues. In some prior existing systems, the surgeon must rely on periodic visual monitoring of pressure to ensure that the catheter is in place. With the use of constant pressure delivery with upper and lower flow rate limits, the instrument microprocessor will immediately detect any change in pressure caused by the dislodged cannula and will compensate by increasing flow rate. When it is “evident” to the microprocessor, through preprogrammed limits or algorithms, that the defined constant pressure cannot be maintained while remaining within the limits of flow rate, the instrument will sound an alarm, alerting the perfusionist and surgeon to the problem. In other situations, when some leakage exists in the connection between the cannula and the blood vessels, increasing the rate of flow may maintain the defined constant pressure so that adequate flow to the tissues is maintained despite the leakage.

In a method of operation of the instrument, at the beginning of a perfusion procedure, the perfusionist will ramp up flow rate by manually adjusting flow rate knob 766 until a desired or predetermined pressure is achieved. After the flow rate is established at a more or less steady state at which a desired or a predetermined pressure is being maintained, then the perfusionist may, in a preferred embodiment, activate an automatic pressure maintenance mode with switch 759. This defines the constant pressure. The flow rate would then be automatically varied, as by control signals from the microprocessor, to keep the existing defined pressure. The upper and lower pressure limits would no longer be appropriate or necessary. The appropriate limits would be those for the flow rate. Upper and lower flow rate limits may be set by the perfusionist or preferably, according to one embodiment of the present invention, may be automatically established based upon a reverse proportionality ratio calculated from the previously existing upper and lower pressure limits and the existing flow rate and defined pressure at the time the automatic constant pressure mode is activated.

In a preferred embodiment, the operation of the pump is controlled to allow automatic pressure maintenance. Operating limits for pressure are selected for both high and low pressure limits. The selection may be made by the operator or may be automatically set by the control system. Normally, the operator gradually increases the flow rate of the pump and observes the resultant pressure. A desired or predetermined operating pressure, for example, 50 mm Hg for retrograde flow, may be established. At this point, if the flow rate meets the criteria that the user expects for an operating pressure of 50 mm Hg, an “automatic” button 759 or a constant pressure button 759 may be pushed. Activation of the “automatic” button is preferably optional so that a perfusionist or a surgeon who is uncomfortable with, or simply not accustomed to, the advantages of a constant pressure blood mixture delivery system can use the delivery system.

Once the automatic button 759 is pushed, the pressure that is displayed at 714 becomes the desired operating delivery pressure and the flow rate begins to vary automatically according to program controls to maintain that pressure. In addition, the alarm limits for the operating system become high and low flow rate limits. Advantageously, these flow rate limits or alarm rates may be calculated and set by using the operating delivery pressure, the flow rate, and the pressure alarm limits that are in effect when the “automatic” button is pushed, i.e., when the change is made to constant pressure operating mode. Calculation of the new limits will be based upon a preprogrammed algorithm of proportionality. For example, if at a particular flow rate of 300 ml/minute, there is an operating pressure of 50 mm Hg and if pressure alarm limits have been set at 20 mm Hg lower limit and 70 mm Hg upper limit, the new proportional flow rate limits might be calculated as follows:

Lower Flow Rate Limit/Lower Pressure Limit=Flow Rate/Pressure

Lower Flow Rate Limit=(Flow Rate/Pressure)×Lower Pressure Limit

=(300 ml/min/50 mmHg)×20 mmHg

Lower Flow Rate Limit=120 ml/min

Upper Flow Rate Limit/Upper Pressure Limit=Flow Rate/Pressure

Upper Flow Rate Limit=(Flow Rate/Pressure)×Upper Pressure Limit

=(300 ml/min/50 mm Hg)×70 mm Hg

Upper Flow Rate Limit=420 ml/min

As long as the pump operates between those flow rate limits, no alarm limit is exceeded. In the event that the rate necessary to maintain pressure exceeds the operating upper limit, there are certain visual operating conditions that make the user aware that the upper limit has been reached.

The upper and lower limits are established to activate various alarm systems that in the preferred embodiment will include a period of flashing displays, such as a flashing upper limit when the upper limit is approached or a flashing lower limit when the lower limit is approached. This might be used in conjunction with an audible alarm. Alternatively, an audible alarm may be initiated after a given time period of warning flashing. Subsequent to a warning alarm in combination with the flashing lights, the system may be turned off and then automatically move into an inactive mode, unless an override switch 767 is activated. The alarm condition may also be depicted on an information/time display screen 769.

The user can change alarm limits if the set alarm limit does not suit the user. One or more algorithm tests are performed automatically, as with preprogrammed computer processing, to be sure that the limit is in fact a continuous or on-going problem. Eventually, if the problem persists and is not merely a transient condition, an alarm may be activated. The rate will not be permitted to go outside of the operating limits, both high and low limits will be imposed and maintained as the operating pressure prior to alarm. An override switch or button may be actuated to abort all limits and to allow the pump to be manually operated. Manual operation, in the preferred embodiment, will mean controlling the output flow rate of the pump by the operator directly through knob 766. Thus, the system is returned to a more traditional mode of operation by actuation of the override switch 767.

Information/time display screen 769 is advantageously included on or adjacent to the same face 740 of display 252. The information/time display screen 769 may include a large LED screen with multiple display fields, such as information display column 771 and 773. The information/time display screen 769 may also be provided in combination with a plurality of soft keys 775, 779, 783 and 787.

Soft key 775 is configured adjacent to, and corresponds to, information field 777. Soft key 779 is correspondingly located adjacent to information field 781. Soft key 783 is adjacent to information field 785. Soft key 787 is adjacent to display field 789. Additional soft keys 791A and 791B are provided for use in connection with optional system configurations.

In the preferred embodiment, there are also designated function or mode keys provided in association with information display screen 769, such as a switch, key or button 703, for entering into a priming mode by which the system is primed with appropriate component solutions, such as a particular timer mode switch 705, a volume function switch 795, a calibration mode switch 707 and a defaults mode switch 797.

Aided by the control offered by the MPS system, the perfusionist is able to keep the amount of cardioplegic solution low, since it is delivered directly to its target, the heart. Without a separate pump to handle the delivery to the heart, much more cardioplegic solution would be necessary to control heart function, which could cause undesired systemic side effects.

Heart surgery is continuing to evolve as new techniques and medications are discovered to lessen the negative effects of the surgery and to impove the therapeutic benefit. Since the earliest surgeries, the pumps have been redesigned so that they do not injure the blood elements they are treating, newer medications have been discovered that lessen the injury to the heart from the necessary ischemic (lacking an inflow of arterial blood) periods, and techniques have been developed to work on a still or beating heart, to name just a few. Drug studies on ischemia have shown that the damage to the heart can be mitigated by the administration of the specific drugs such as adenosine or cariporide to the myocardial system. See, for example, “Broad-Spectrum Cardioprotection With Adenosine”, Vinten-Johansen et al., presented at the International Symposium on Myocardial Protection from Surgical Ischemic-Reperfusion Injury, Asheville, N.C., Sept 21-24, 1997; “Adenosine-Supplemented Blood Cardioplegia Attenuates Postichemic Dysfunction After Severe Regional Ischemia”, Thourani et al., Circulation, Vol. 100, No. 19, November 1999; “Adenosine Myocardial Protection: Preliminary Results of a Phase II Clinical Trial”, Mentzer et al., Annals of Surgery Vol. 229, No. 5, 643-650, 1999.

However, at least two problems present themselves with the administration of drugs such as adenosine. Adenosine acts as a systemic vasodilator, relaxing the muscles of the vascular system, and lowering the blood pressure, sometimes drastically. Secondly, the half-life of adenosine is very brief once it has been exposed to the blood, only 13 seconds. Since the lines between the body and the equipment handling the blood is several yards long, it is difficult to deliver such a drug both economically and in the precise concentrations necessary to provide the benefit to the heart or other targeted organ without causing undesirable systemic side effects. With these limitations, the benefits of these drugs have not been realized It would be very desirable to be able to administer adenosine, as well as similar medications that react with the blood, directly to the target organ, without mixing with the blood until absolutely necessary.

### SUMMARY OF THE INVENTION

The present invention provides a method and apparatus for delivering precisely measured medications to a targeted area of the body, such as the coronary arteries or coronary sinus, while delaying the contact between the medication and the blood until necessary. In this invention, the primary pump is routed as previously through a first conduit to the target organ, while the output of the second pump is sent through a second conduit that parallels the path of the first conduit, but remains separate from it. These conduits can be separate lines or a single line having two separate lumens. At a point close to the target area, the two lines merge into a single line that is connected to the target organ or area. In the presently preferred embodiment, the lines merge at a point not more than twelve inches from the target organ. The necessary precise measurements are handled by the MPS system, yet the medication is not subject to lengthy contact with the blood prior to reaching its target.

### BRIEF DESCRIPTION OF THE DRAWINGS

The novel features believed characteristic of the invention are set forth in the appended claims. The invention itself, however, as well as a preferred mode of use, further objectives and advantages thereof, will best be understood by reference to the following detailed description of an illustrative embodiment when read in conjunction with the accompanying drawings, wherein:

FIG. 1 shows a diagrammatic representation of the connections to the heart during open-heart surgery using a heart-lung machine and an MPS to handle blood flow.

FIG. 2 depicts the parts of a prior art cardioplegia delivery system.

FIG. 3 shows the disposable pump cassette used in the MPS system.

FIG. 4 shows a prior art pump mechanism from an MPS system.

FIG. 5 shows another view of the pump mechanism of FIG. 4.

FIG. 6 shows the inlet and the outlet valve arrangement from a single pump chamber.

FIG. 7 is a detailed perspective view of a control panel from an exemplary MPS system.

FIG. 8 shows a close-up of a portion of the control panel of FIG. 7.

FIG. 9 shows a schematic drawing of a delivery system according to one embodiment of the invention,

FIGS. 10A-C show a delivery system according to an alternate embodiment of the invention.

### DETAILED DESCRIPTION OF THE INVENTION

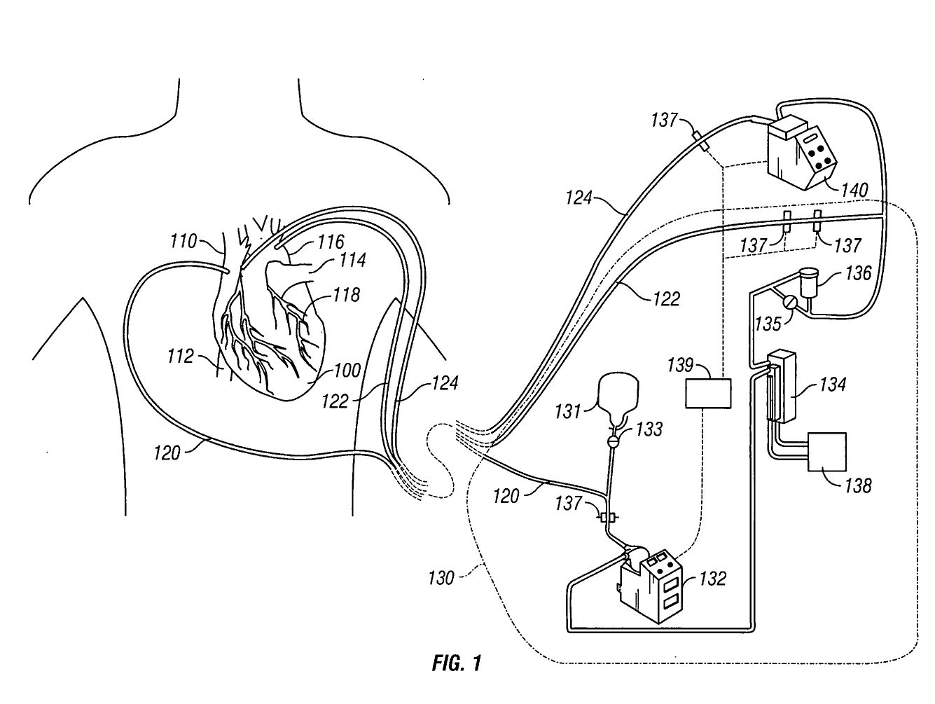

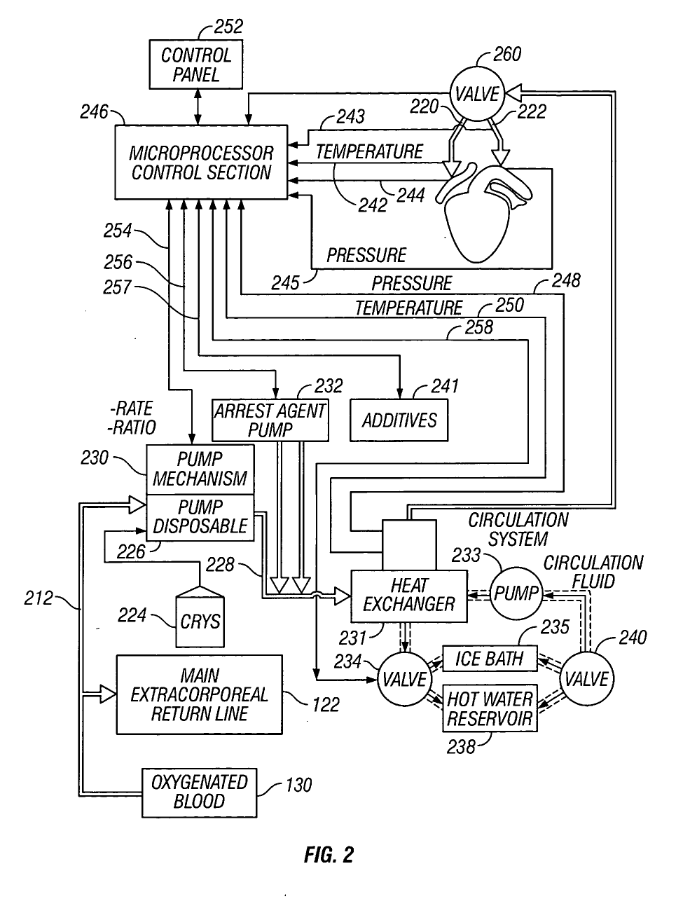

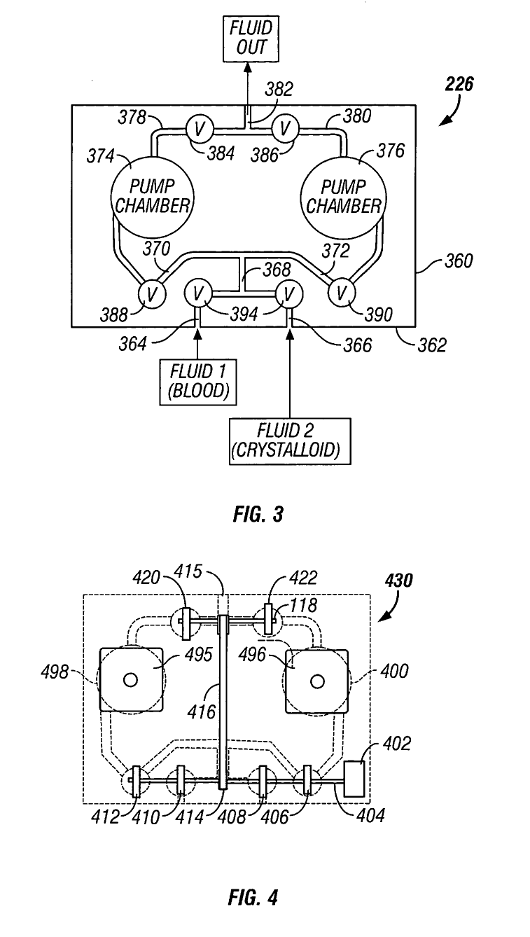

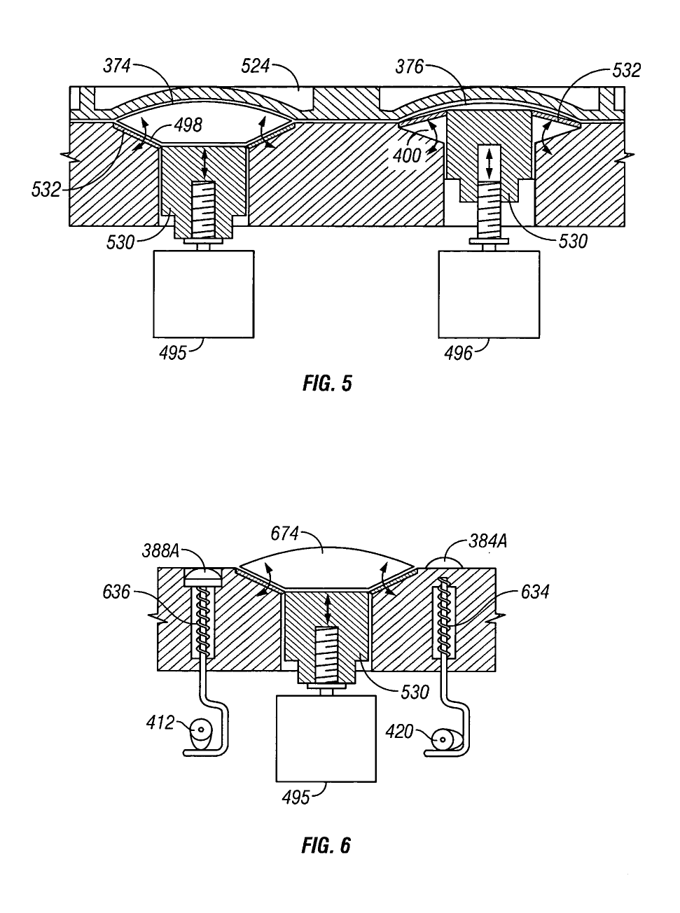

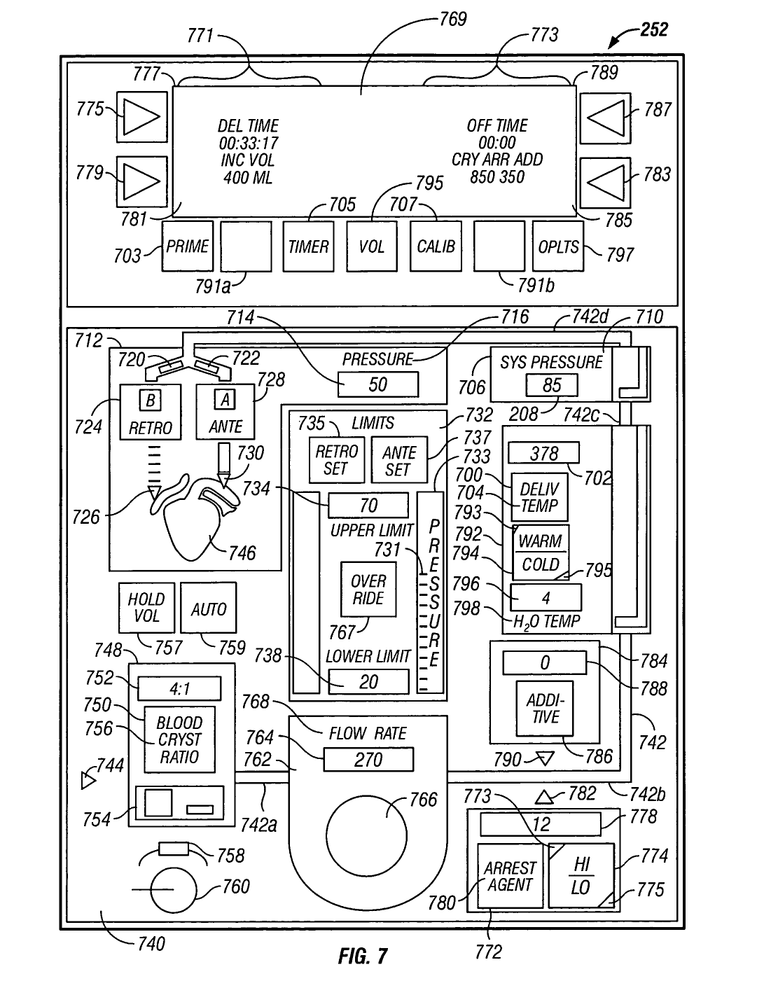

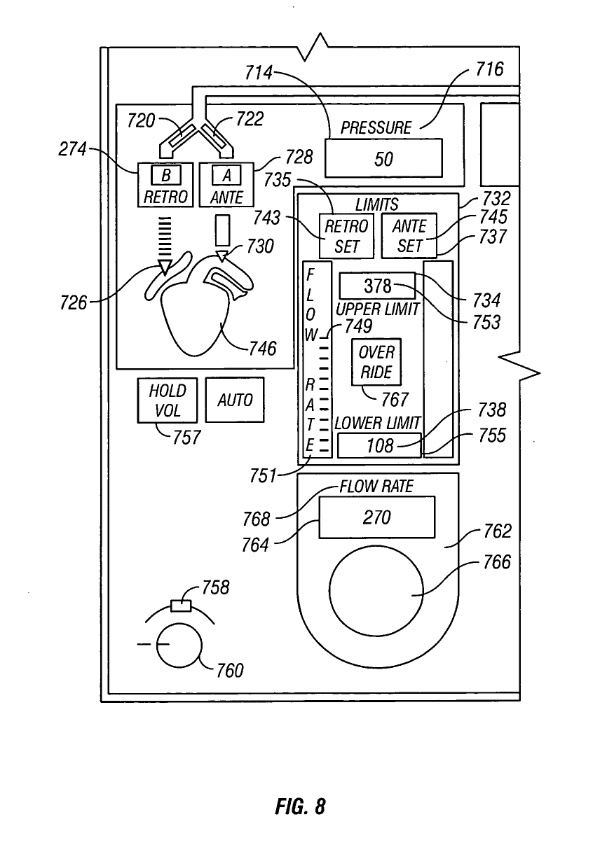

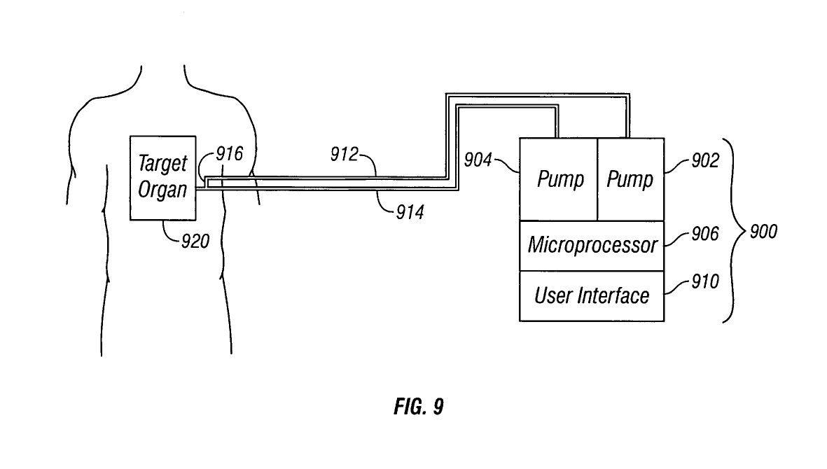

A first embodiment of the disclosed invention will be described with reference to FIG. 9. This drawing is a simplification of the drawing of FIG. 1, showing only the MPS system 900 and the associated delivery lines 912, 914 to the target organ 920, which in this example is the vascular system serving the heart, either the coronary arteries or the coronary veins through the coronary sinus. The MPS system 900 can be similar to the MPS system 140 of FIG. 1 or can be an alternative delivery system. Shown are the user interface 910, through which the perfusionist monitors activity within the system and provides control input, microprocessor 906, which interprets signals from monitors within the system, sends data to the user interface, receives input from the user interface, and send signals to controllers within the system to implement user commands, and two pumps 902, 904. Pump 904 is the main pump in this embodiment. This pump receives blood from the heart-lung machine (not specifically shown), to which it adds crystalloid solution, as requested by the surgeon and perfusionist, then pumps the blood mixture through line 914 to the target organ of the patient. When requested, a cardioplegic solution will be metered by an additional pump (not shown) and added to the delivery line just downstream of the pump 904. Pump 902 is used to pump adenosine in this embodiment. The adenosine is separately metered into line 912, which is also directed to the target organ 920. These delivery lines 912, 914 are bundled with the delivery lines carrying venous blood from the patient and arterial blood being returned to the patient, as was shown in FIG. 1. Line 914 only is attached to deliver blood containing the necessary cardioplegic solution to the myocardial system. As the two lines 912, 914 approach the attachment to the vessels serving the myocardial system, the delivery line 912 that contains adenosine is joined to delivery line 914 at junction 916, so that the two fluids enter the target organ at the same time. In the presently preferred embodiment, the junction 916 of delivery line 914 with 912 is no more than 12 inches from the target organ. This allows minimal time for the adenosine to be broken down by contact with the blood before it is delivered to the site where it is needed. In alternate embodiments, this maximum distance can be greater or less, depending on the particular components in the delivery lines and their reactivity with each other. Because the adenosine in this example can be delivered so precisely and with so little blood contact before the heart, this opens the door for its use in heart surgery, where previously this was not possible to implement. Additionally, low doses can be used, as only the targeted organ receives the medication-containing blood, which is then returned to the heart-lung machine. Systemic effects are kept low. Additionally, the cost of the additional delivery line 912 is minimal.

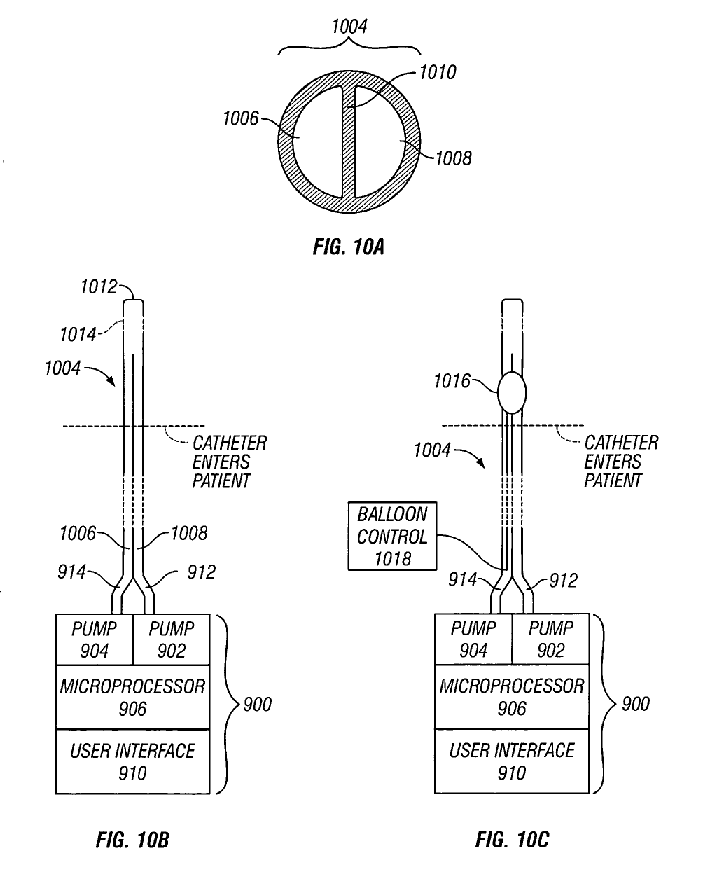

An alternate embodiment of the invention illustrated in FIGS. 10A through 10C. In this embodiment, the delivery lines 912, 914 are implemented in a catheter 1004 having two lumen, or bores. A cross-section of catheter 1004 is shown in FIG. 10A. Catheter 1004 has a partition 1010, which divides the interior longitudinally into separate lumen 1006, 1008. This eliminates the need for two separate lines, which must be joined. In FIG. 10B, MPS system 900 is shown with output lines 912, 914. Pump 904 of MPS system 900 pumps blood with a cardioplegic agent into delivery line 914, while pump 902 pumps adenosine into delivery line 912. Before these lines join the other bundled lines to traverse the distance to the patient, delivery lines 912, 914 enter catheter 1004, where they remain unmingled in separate lumen. In this example, the catheter is inserted into the circulatory system of the patient, e.g., into an artery, where the catheter can be maneuvered into the aortic root, adjacent the coronary arteries. At a location near the tip 1012 of the catheter 1004, openings 1014 in the outer catheter wall allow the blood and adenosine to be released from catheter 1004. In this example, partition 1010 does not extend to the end of catheter 1004, but terminates just prior to the openings 1014, so that the adenosine is mixed with the blood just before it leaves the catheter.

FIG. 10C shows a similar embodiment, except that a balloon 1016 is attached to catheter 1004. Once the catheter 1004 is correctly positioned in the patient, the balloon 1016 is inflated by means of balloon controller 1018, so that passage of the blood and medications delivered through catheter 1004 are prevented from flowing back into the portion of the vessel in which the catheter is inserted.

The description of the present invention has been presented for purposes of illustration and description, and is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated.

We claim:

1. A clinical fluid pumping system comprising:
a first pump that is configurable to pump a first metered amount of a first fluid through a first delivery line to a catheter;
a second pump that is configurable to pump a second metered amount of a second fluid through a second delivery line, separate from said first delivery line, to said catheter;
a processor, connected to control said first and said second pumps such that said second metered amount has a definable relationship to said first metered amount;
wherein the lumen of said first delivery line and the lumen of said second delivery line remain separate up to a connection point of said first and second delivery lines to said catheter.

2. The clinical fluid pumping system of claim 1, wherein the first fluid is an oxygen-carrying solution.

3. The clinical fluid pumping system of claim 1, wherein the first fluid is blood.

4. The clinical fluid pumping system of claim 1, wherein the first fluid comprises blood from the patient.

5. The clinical fluid pumping system of claim 1, wherein the second fluid is adenosine.

6. The clinical fluid pumping system of claim 1, further comprising a plurality of additional pumps pumping respective fluids under the control of said processor.

7. The clinical fluid pumping system of claim 1, wherein said first pump is further configurable to combine a third metered amount of a third fluid with the first fluid and to pump both the first and the third fluids into said first delivery line.

8. The clinical fluid pumping system of claim 1, wherein the fluids are delivered at a controlled temperature and pressure.

9. The clinical fluid pumping system of claim 1, wherein said processor receives feedback from monitors and can automatically alter operational parameters to meet predefined objectives.

10. The clinical fluid pumping system of claim 1, wherein an operator can alter the definable relationship between said first metered amount and said second metered amount.

11. The clinical fluid pumping system of claim 1, wherein the second delivery line contains a one-way check valve to prevent retrograde flow.

12. The clinical fluid pumping system of claim 1, further comprising a temperature controller, configurable to provide heating or cooling to fluids in at least one of said first delivery tube and said second delivery line without contaminating the fluids.

13. The clinical fluid pumping system of claim 1, further comprising a monitor to detect one or more conditions, the conditions including the rate of flow, the temperature, the pressure, and the concentration of a fluid within said pumping system.

14. The clinical fluid pumping system of claim 1, further comprising a monitor to detect the temperature or blood pressure of a patient coupled to said pumping system.

15. The clinical fluid pumping system of claim 1, wherein said processor is connected to control the activity of a portion of said clinical fluid pumping system.

16. The clinical fluid pumping system of claim 15, wherein said processor is connected to control the operation of a valve.

17. The clinical fluid pumping system of claim 15, wherein said processor is connected to control the speed of said first pump.

18. The clinical fluid pumping system of claim 1, wherein said processor is connected to receive inputs from a monitor and to send signals to control a portion of said clinical fluid pumping system.

19. The clinical fluid pumping system of claim 1, further comprising a display and control panel connected to provide information regarding the operation of said pumping system to a user and to accept input from the user.

20. The clinical fluid pumping system of claim 1, wherein said first delivery line and said second delivery line are separate lumen within a single tubing.

21. The clinical fluid pumping system of claim 1, wherein said first delivery line and said second delivery line are separate pieces of tubing.

22. The clinical fluid pumping system of claim 1, wherein the second fluid is co-mingled with the first fluid no further from a target organ than 12 inches.

23. The clinical fluid pumping system of claim 1, wherein said catheter is directly inserted into a circulatory vessel serving a target organ and has a single lumen.

24. The clinical fluid pumping system of claim 1, wherein said catheter is inserted into a circulatory vessel remote from a target organ and maneuvered to the target organ, said catheter having multiple lumen.

25. A clinical fluid pumping system, comprising:
a first delivery line for receiving a first fluid;
a second delivery line for receiving a second fluid; and
pumping means connected to advancing the first and second fluids through their respective said delivery lines in a known relationship to each other;
wherein said first delivery line and said second delivery line prevent the co-mingling of the first fluid and the second fluid up to a connection point of said first and second delivery lines to a catheter.

26. The clinical fluid pumping system of claim 25, wherein said pumping means consists of a first pump.

27. The clinical fluid pumping system of claim 25, wherein said pumping means comprises a first pump and a second pump.

28. The clinical fluid pumping system of claim 25, wherein the co-mingling of the first fluid and the second fluid is delayed to prevent degradation of the second fluid.

29. The clinical fluid pumping system of claim 25, further comprising a heat exchanger that is connected to provide heating or cooling to the fluids in said first and second delivery lines without contaminating the fluids.

30. The clinical fluid pumping system of claim 25, further comprising means to control the fluid flow rate.

31. The clinical fluid pumping system of claim 25, further comprising a means to control the known relationship between the first and the second fluids.

32. The clinical fluid pumping system of claim 25, further comprising a monitor to detect the temperature or pressure of the fluid within said pumping system

33. The clinical fluid pumping system of claim 25, further comprising a monitor to detect the temperature or blood pressure of a patient coupled to said pumping system.

34. The clinical fluid pumping system of claim 25, further comprising a processor connected to control the activity of a portion of said clinical fluid pumping system.

35. The clinical fluid pumping system of claim 34, wherein said processor is connected to control the operation of a valve.

36. The clinical fluid pumping system of claim 34, wherein said processor is connected to control the speed of said first pump.

37. The clinical fluid pumping system of claim 25, further comprising a processor connected to receive inputs from a monitor and to send signals to control a portion of said clinical fluid pumping system.

38. The clinical fluid pumping system of claim 25, further comprising a display and control panel connected to provide information regarding the operation of said pumping system to a user and to accept input from the user.

39. The clinical fluid pumping system of claim 25, wherein the first fluid comprises blood and the second fluid comprises adenosine.

40. The clinical fluid pumping system of claim 25, wherein said first delivery line and said second delivery line are separate lumen of a single catheter.

41. The clinical fluid pumping system of claim 25, wherein the second fluid is co-mingled with the first fluid no further from the delivery site on the patient than 12 inches.

42. A method of providing a therapeutic agent to a targeted portion of the vascular system of a patient, said method comprising the steps of:
providing a fluid pumping system;
receiving a supply of a first fluid into said fluid pumping system;
pumping the first fluid at a measured rate to a first delivery line attached to a catheter; and
metering a given amount of a second fluid to a second delivery line for delivery to said catheter, wherein the second fluid is delivered at a rate that is tied to the delivery rate of the first fluid;
wherein the first fluid in said first delivery line and the second fluid in said second delivery line are not co-mingled at least until delivery into said catheter.

43. The method of claim 42, further comprising the step of:
passing at least one of the fluids through a heat exchanger, whereby the at least one of the fluids is brought to a desired temperature prior to delivery to said catheter.

44. The method of claim 42, further comprising the step of:
monitoring the temperature or pressure of a fluid within said fluid pumping system.

45. The method of claim 42, further comprising the step of:
attaching a monitor to detect the temperature or blood pressure of a patient connected to said pumping system.

46. The method of claim 42, further comprising the step of:
using a processor to control the activity of a portion of said fluid pumping system.

47. The method of claim 46, wherein said processor controls a valve.

48. The method of claim 46, wherein said processor controls the speed of a first pump in said pumping system.

49. The method of claim 46, wherein said processor receives inputs from a monitor and automatically controls a portion of said fluid pumping system.

50. The method of claim 42, further comprising the step of:
providing information regarding the operation of said pumping system to a display and accepting input from a user.

51. The method of claim 42, wherein said receiving step receives a fluid comprising blood and said pumping step pumps adenosine.

52. The method of claim 42, wherein said catheter is directly inserted into a circulatory vessel serving a target organ and has a single lumen.

53. The method of claim 42, wherein said catheter is inserted into a circulatory vessel remote from a target organ and maneuvered to the target organ, said catheter having multiple lumen.

54. The method of claim 42, wherein the second fluid is co-mingled with the first fluid no further from a target organ than 12 inches.

55. The method of claim 42, further comprising at least one additional pump to pump a predetermined amount of a third fluid into said first delivery line.

56. The method of claim 42, wherein the fluids are delivered at a controlled temperature and pressure.

57. The method of claim 42, wherein an operator can alter the relationship between the delivery rate of the first fluid and the delivery rate of the second fluid.

58. The method of claim 42, further comprising the step of providing a check valve in sad second delivery line to prevent retrograde flow of fluids in said second delivery line.
